(19) United States
(12) Patent Application Publication
(10) Pub. No.: US 2017/0303557 A1
(43) Pub. Date: Oct. 26, 2017

(54) PURIFICATION OF POLYPHENOLS

(71) Applicant: NESTEC S.A., Vevey (CH)

(72) Inventors: Francoise Le Derff, Morges (CH); Arne Glabasnia, Murten (CH); Eric Dossin, Jougne (FR); Delphine Musset, Derby Derbyshire (GB); Valerie Martine Jeanine Leloup, Orbe (CH)

(21) Appl. No.: 15/517,663

(22) PCT Filed: Oct. 6, 2015
(86) PCT No.: PCT/EP2015/072978

§ 371 Date: Apr. 7, 2017

(30) Foreign Application Priority Data
Oct. 9, 2014 (EP) 14188365.2

(51) Int. Cl. A23F 5/02; A23L 33/105; A23L 2/56; A61K 31/352

(52) U.S. Cl. CPC A23F 5/02; A61K 31/352; A23L 33/105; A23L 2/56; A23V 2250/2132

(57) ABSTRACT

The present invention relates to a A process for providing a fraction enriched in polyphenols from a starting material, the process comprises the steps of: (i) solubilizing the starting material in an aqueous solvent; (ii) adjusting pH to below pH 3 (preferably about pH 1); (iii) contacting the starting material with a chromatographic resin; (iv) desorbing the polyphenols from the chromatographic resin to provide an eluate comprising the polyphenols; (v) adjusting pH of the eluate to above pH 3.5 (preferably pH 4); and (vi) separating the eluate from the eluted polyphenols to obtain the fraction enriched in polyphenols. The invention furthermore related to products comprising such polyphenols enriched fractions.

### TECHNICAL FIELD OF THE INVENTION

The present invention relates to a process for the purification of polyphenols from a starting material. In particular the present invention relates to a fraction enriched in polyphenols, in particular chlorogenic acids (CGA's) obtainable from a plant material, wherein the isomer balance of the enriched fraction is substantially maintained compared to the starting material.

### BACKGROUND OF THE INVENTION

An important property of beverages, such as coffee is persistent foam also referred to as “crema” and intense aroma which are considered important quality criteria. The volume, texture, finesse, color and stability of the crema are distinctive characteristics appealing to the consumer. Crema results from the extraction of surface active coffee components that coat and stabilize the gas bubbles created by blasting the tamped espresso coffee matrix with pressurized heated water.

Coffee aroma is also an important property because the intensity of the coffee aroma is the first sensoric experience that meets the consumer. When smelling coffee, the aroma can help you evaluate the coffee flavor and the brightness of the coffee. During roasting many aromatic compounds are formed or liberated from the coffee bean matrix and affect the experience (aroma and flavor) of the coffee. One of the most important precursor of aromatic compounds are chlorogenic acids, in particular caffeoyl quinic acid.

Thus, there has been an interest to isolate a polyphenol enriched fraction from many different kind of plant materials, e.g. berries, grapes, citrus, vegetables, cereals, herbs, tea, coffee, cocoa. In particular there has been an interest to isolate a polyphenol fraction enriched in chlorogenic acid compounds and which has an isomeric balance of the chlorogenic acid compounds that resemples the original plant material, in order to spike food and beverage products with e.g. aroma and flavor.

Furthermore, polyphenols have been reported to have antimicrobial, antiviral, antimutagenic, anticarcinogenic, antiproliferative and vasodilatory effects. Polyphenols may have antibacterial activity which may be useful in the combat of tooth decay caused by Streptococcus mutans. Polyphenols are listed as nootropics purported to improve mental functions such as cognition, memory, intelligence, motivation, attention and concentration.

However, strengthening of the rules for health claim in food legislation makes it compulsory to ensure a consistent level in the active ingredient as well as to refer to scientific evidence obtained on the manufactured products.

Thus, there is a need for a process for providing a polyphenol enriched fraction, especially comprising chlorogenic acids (CGA's) where the chlorogenic acid balance resembles the chlorogenic acid balance originally present in the plant material. The enriched fraction may subsequently be used to ensure a constant level in active ingredient of food product as well as in intervention studies to demonstrate the cause and effect relationship between the intake of polyphenols, such as from coffee, and a given claimed effect.

Thus, there is a need in the industry to provide a process for providing a fraction enriched in polyphenols, in particular chlorogenic acid, having the most authentic characteristics relative to the plant material it originates from which may be used to enhance the properties of a food product or a beverage.

### SUMMARY OF THE INVENTION

Accordingly, the present invention describes a novel process for the purification of polyphenols where the polyphenolic balance is maintained or substantially maintained. Furthermore, the process of the present invention may result in a limited or no formation of derivatives formed during the process e.g. ethylesters of the polyphenols. Ethylesters are undesirable because they do not have the same properties as the native polyphenols naturally occurring in the plant material. In addition, ethylesters change the polyphenol balance of the enriched fraction relative to the original product.

Thus, one aspect of the invention relates to a process for providing a fraction enriched in polyphenols from a starting material, the process comprises the steps of:
(i) solubilizing the starting material in an aqueous solvent;
(ii) adjusting pH to below pH 3;
(iii) contacting the starting material with a chromatographic resin;
(iv) desorbing the polyphenols from the chromatographic resin to provide an eluate comprising the polyphenols;
(v) adjusting pH of the eluate to above pH 3.5; and
(vi) separating the eluate from the eluted polyphenols to obtain the fraction enriched in polyphenols.

Another aspect of the present invention relates to a fraction enriched in polyphenols obtainable from the process according to the present invention.

Yet another aspect of the present invention is to provide a food ingredient comprising the fraction enriched in polyphenols according to the present invention.

Still another aspect of the present invention is to provide a food product comprising the fraction enriched in polyphenols according to the invention and/or the food ingredient according to the present invention.

A further aspect relates to the use of the fraction enriched in polyphenols according to the present invention and/or the food ingredient according to the present invention as a precursor for generating aroma, flavor and/or foaming.

Yet another aspect relates to a pharmaceutical composition comprising the fraction enriched in polyphenols according to the present invention and a pharmaceutical acceptable carrier.

### BRIEF DESCRIPTION OF THE FIGURES

FIG. 1 shows a non-limiting example of polyphenols of the present invention. The (*) marking in the figure represents the connection point of R¹.

FIG. 2 shows desorption kinetics with 90% ethanol for CQAs, FQAs, diCQAs and caffeine.

FIG. 3 shows a specific embodiment relating to a purification process according to the present invention at labscale.

FIG. 4 shows the global composition of polyphenol enriched green coffee fraction as compared to initial coffee.

FIG. 5 shows CGA composition of polyphenol enriched green coffee fraction as compared to initial coffee.

FIG. 6 shows degradation of 5-CQA in 75% ethanol after 2 h at pH 1, 2, 7.4 and 12.

FIG. 7 shows transformation of 5-CQA to its ethylester at t=RT, 50° C. and 80° C.

FIG. 8 shows Formation of 5-CQA ester (a) at 50° C. within 3 hours and (b) at RT within 36 hours.

FIG. 9 shows an improved purification protocol for production scale.

FIG. 10 shows the global composition of decaffeinated polyphenol enriched green coffee fraction as compared to initial coffee for clinical studies.

FIG. 11 shows the detailed CGA composition of enriched decaffeinated fraction as compared to initial coffee.

FIG. 12 shows the global composition of caffeinated polyphenol enriched green coffee fraction as compared to initial coffee for clinical studies.

FIG. 13 shows the detailed CGA composition of enriched caffeinated fraction as compared to initial coffee.

FIG. 14 shows the purification protocol for chlorogenic acids using successive desorption steps with increasing ethanol ratio in desorption mixtures (1) 20%, (2) 50% (3) 80%.

FIG. 15 shows the relative composition of CGAs in initial coffee and the three fractions obtained by ethanol gradient desorption.

FIG. 16 shows HPLC chromatograms of the three different ethanol gradient desorption fractions.

The present invention will now be described in more detail in the following.

### DETAILED DESCRIPTION OF THE INVENTION

The present invention describes a novel process for providing a fraction enriched in polyphenols using a chromatographic resin. The enriched fraction obtained from the present invention may provide improved properties, in particular aroma, flavour and foaming properties, since the isomer balance of the polyphenols (such as chlorogenic acid compounds) present in the enriched fraction is substantially maintained and resemble the isomer balance of the polyphenols (such as chlorogenic acid compounds) originally present in the plant material. Furthermore, the process for providing a fraction enriched in polyphenols according to the present invention also provides an enriched fraction with a high purity and/or good properties (product performance).

In an embodiment of the present invention the enriched fraction has a content of chlorogenic acid compounds of at least 40% (w/w), such as at least 50% (w/w), e.g. at least 60% (w/w), e.g. at least 70% (w/w).

The presented process may have several advantages compared to other purification processes. One advantage of the present invention may be that the isomeric balance of polyphenols, in particular, chlorogenic acid compounds, of the enriched fraction may be maintained. Another advantage may be that the process generates limited or no ethylesters of polyphenols. A further advantage of the present invention may be that a high level of purity (i.e. >60%) may be obtained.

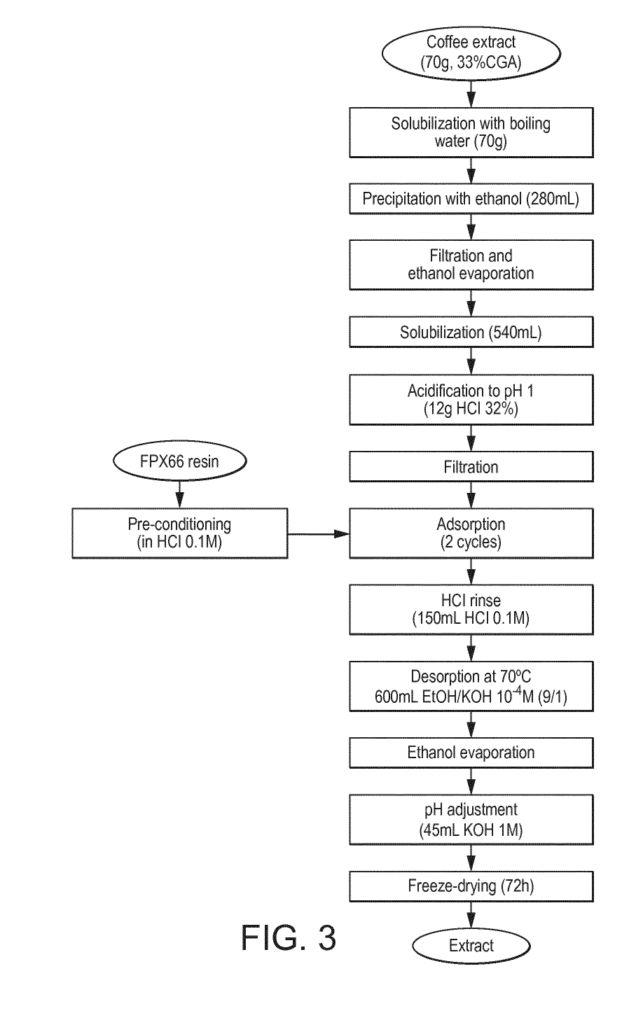

Schematic details of the process according to the invention are displayed in FIGS. 3, 9 and 14. Each scheme represents specific embodiments of the process according to the invention.

Thus, an aspect of the present invention relates to a process for providing a fraction enriched in polyphenols from a starting material, the process comprises the steps of:
(i) solubilizing the starting material in an aqueous solvent;
(ii) adjusting pH to below pH 3;
(iii) contacting the starting material with a chromatographic resin;
(iv) desorbing the polyphenols from the chromatographic resin to provide an eluate comprising the polyphenols;
(v) adjusting pH of the eluate to above pH 3.5; and
(vi) separating the eluate from the eluted polyphenols to obtain the fraction enriched in polyphenols.

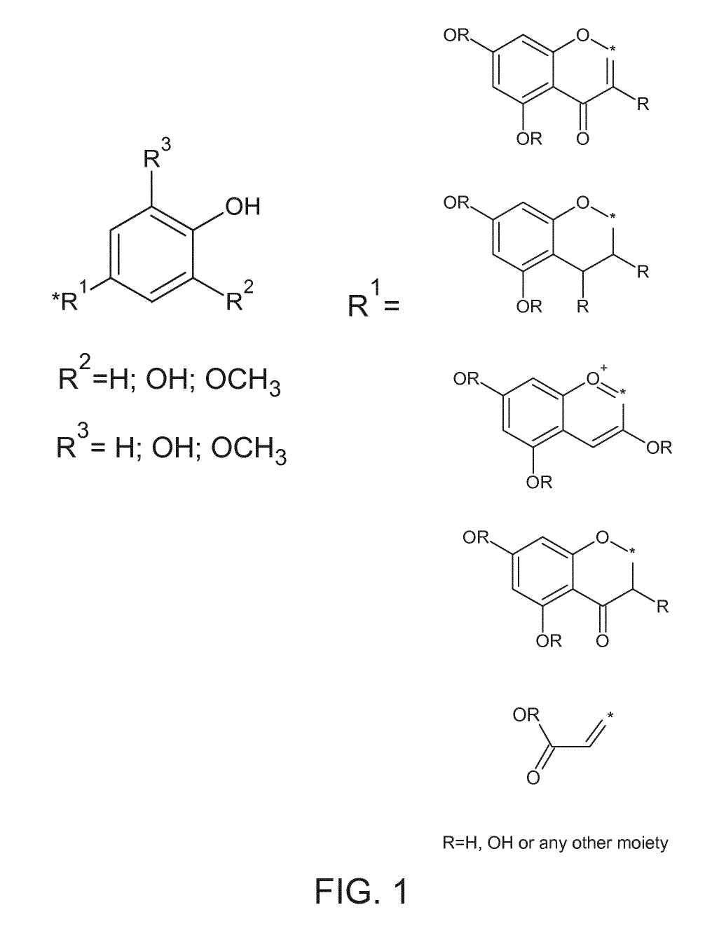

This process was developed based on the chemical and physical properties of polyphenol compounds, for example polyphenol compounds as described in FIG. 1. One of the most important polyphenols are chlorogenic acids (CGA's) which are the most dominant polyphenols found in coffee, as well as many other plant materials. Specific (non-limiting) examples of chlorogenic acids (CGA's) are 3-caffeoyl quinic acid (3CQA), 4-caffeoyl quinic acid (4CQA), 5-caffeoyl quinic acid (5CQA), 3,4-dicaffeoyl-quinic acid (3,4diCQA), 3,5-dicaffeoyl-quinic acid (3,5diCQA), 4,5-dicaffeoyl-quinic acid (4,5diCQA), 3-feruloyl quinic acid (3FQA), 4-feruloyl quinic acid (4FQA), 5-feruloyl quinic acid (SFQA), and caffeic acid.

Chlorogenic acids are low molecular weight compounds, globally below 600 g/mol. Chlorogenic acids bear one or several aromatic groups (i.e. phenolic moiety). Non-covalent aromatic interactions, also called π-π stacking interactions, can be established between compounds containing these aromatic moieties. This property is used to specifically adsorb chlorogenic acids to matrices bearing aromatic groups.

Chlorogenic acids bear a quinic acid group which confers acid-base properties (pKa=3.2). At pH below pKa, the chlorogenic acid is protonated. The molecule is uncharged and therefore it is rather hydrophobic. At pH above the pKa, the acid function is under a carboxylate form, which means that the entire molecule is more hydrophilic due to the charge. This property can be used to modulate the hydrophobicity of the molecule for purification purposes.

The starting material comprising the polyphenols may be of different origin. In an embodiment the starting material is a plant material. In yet an embodiment the plant material is selected from the group consisting of coffee, such as green coffee, caffeinated coffee, decaffeinated coffee and decaffeinated green coffee, malt, cocoa, tea, berries, grapes, vegetable, citrus, herbs and cereals. The starting material may initially be solubilized e.g. in boiling water. It may be advantageous to perform an initial precipitation step of the solubilized starting material before starting the above described purification process in order to remove high molecular weight (HMW) compounds like proteins, melanoidins and polypsaccharides e.g. arabinogalactans. Thus, in an embodiment the solubilized starting material obtained in step (i) may be subjected to precipitation e.g. with an alcohol (preferably ethanol) to provide a solid phase and a liquid phase, before the pH-adjustment in step (ii). In yet an embodiment the solid phase may be separated from the liquid phase by filtration, centrifugation and/or decantation, before the pH-adjustment in step (ii). Following the optional separation step the liquid phase may be subjected to an evaporation treatment to remove the alcohol used for precipitating HMW's from the liquid phase, before the pH-adjustment in step (ii).

The fraction enriched in polyphenols may be of different forms. Thus, in an embodiment the fraction enriched in polyphenols may be in liquid form or in dried form. In a further embodiment the fraction enriched in polyphenols is freeze dried.

Example 1 below discloses a particular example, where the solubilized starting material is precipitated with 80% alcohol. Thus, in an embodiment the alcohol is ethanol. In a further embodiment the alcohol such as ethanol has a concentration in the range 50-99% (w/w), such as 60-99% (w/w), such as 60-90% (w/w), such as 70-90% (w/w), such as 75-85% (w/w).

The process according to the present invention includes acidification of the starting material to below pH 3 before it is loaded on to the resin column. In an embodiment of the present invention the pH adjustment in step (ii) is an adjustment to below pH 2.5, preferably below pH 2, more preferably below pH 1.5, or even more preferably to about pH 1. In another embodiment the acid used is selected from the group consisting of strong acid e.g. hydrochloric acid, sulfuric acid and phosphoric acid. The reason for the relatively low pH is that pure water and/or use of higher pH-values may cause desorption of CGA's from the resin. On the other hand, under acidic condition, as chlorogenic acids remain in their protonated and hydrophobic form, interactions with the resin is maintained.

Example 2 presented below demonstrates the effect of lowering the pH to pH 1 before loading the starting material onto the chromatographic resin. After pH adjustment, and before loading the starting material onto the chromatographic resin, the material may be filtered to remove larger debris. The chromatographic resin may also be pre-conditioned with an acid before applying the starting material to be purified. Thus, in an embodiment the chromatographic resin is pre-conditioned with an acid at a pH below 3, preferably below pH 2, more preferably below pH 1.5, or even more preferably to about pH 1. In another embodiment the acid used for pre-conditioning the column is selected from the group consisting of hydrochloric acid, sulfuric acid and phosphoric acid. The exact concentration of the acid may vary. Thus, in an embodiment the concentration of the acid is in the range 0.001M-1M, preferably 0.001M-0.5M, or even more preferably in the range 0.05M-0.3M.

After loading the starting material onto the chromatographic resin it may be advantageous to rinse the chromatographic resin to remove unbound materials. Thus, in an embodiment the chromatographic resin may be treated with a rinsing solution before the polyphenols are desorbed. Preferably, the rinsing solution has a pH value below pH 3, preferably below pH 2, more preferably below pH 1.5, even more preferably about pH 1. In an embodiment the washing solution comprises an acid, such as hydrochloric acid, sulfuric acid or phosphoric acid. As the exact concentration of the acid may vary it is an embodiment of the present invention the concentration of the acid in the rinsing solution may be in the range 0.001M-1M, preferably 0.001M-0.5M, or even more preferably in the range 15 0.05M-0.3M.

Example 3 shows the effect of rinsing the column with 0.1M HCl, resulting in an increase in the purity of the final fraction from 50% to 66% compared to when no rinsing step is included. Thus, it may be concluded that rinsing the column before desorption may further improve the purity of the final fraction.

The ratio between the starting material and the chromatographic material may influence the amount of purified product. Overloading the column may cause the column to be saturated which may result in insufficient isolation of valuable polyphenolic compounds because a fraction of the solubilized starting material may not be adsorbed to the column. Thus, in an embodiment of the present invention the ratio between the starting material and the chromatographic material may be from 2:1 to 1:4 (on a weight:weight basis), such as from 1:1 to 1:3 (on a weight:weight basis), e.g. about 1:2 (on a weight:weight basis). In the present context the term “chromatographic resin” relates to the chromatographic media present in the chromatographic column and responsible for the separation of the starting material. In an embodiment of the present invention the chromatographic resin may be packed in a packed bed or in an expanded bed. Different types of chromatographic resins may be used. Thus in an embodiment of the present invention step (iii) may involve adsorption chromatography on a polymer resin and/or any other chromatographic media known to the skilled person.

For desorption, the interactions between the chromatographic resin and phenolic compounds of the polyphenols have to be weakened. Different parameters may influence the desorption step of the present invention.

The inventors of the present invention found that temperature may be a parameter that has a significant influence on the undesirable formation of ethylesters of the polyphenols in the presence of a hydroxyl component, e.g. ethanol.

Thus, in an embodiment of the present invention the temperature during desorption may be below 70° C., such as below 60° C., such as below 50° C., such as below 40° C. In another embodiment the temperature is in the range 10-40° C. such as in the range of 25-35° C., e.g. at about 30° C. Example 7 demonstrates the advantage of keeping the temperature down to avoid ethylester formation of the CGA's. Thus, it may generally be an advantage to keep the temperature as low as possible during every step of the process. However, it is always a tradeoff between the speed of the assay (e.g. during an evaporation and/or a desorption step) and avoiding the generation of ethylesters.

Another parameter that may influence the desorption process is the composition of the desorption eluent. The desorption eluent may comprise different constituents that may improve desorption of the polyphenols. In an embodiment of the present invention the desorption eluent may comprise a hydroxyl component, preferably the hydroxyl component is ethanol. Other alcohols or combination of alcohols may also be used as desorption eluent.

The inventors of the present invention also found that the pH of the eluate and the desorption eluent may have a significant influence on the undesirable formation of ethylesters of polyphenols during the process in the presence of a hydroxyl compound, e.g. ethanol. In an embodiment of the present invention the pH of the eluate and/or the desorption eluent may be above pH 3, such as above pH 3.5, preferably about pH 4, or even more preferably in the pH range 4-5. At high pH values, e.g. strong alkaline conditions like pH 12, the undesired formation of ethyldesters of chlorogenic acids may be reduced, but may cause other undesirable reactions of the polyphenols.

In yet an embodiment of the present invention the desorption eluent may comprise a hydroxyl component, preferably ethanol, in combination with an aqueous base, the aqueous base may preferably be KOH.

In an embodiment of the present invention the desorption eluent may comprise at least 20% of the hydroxyl component, e.g. at least 30% of the hydroxyl component, such as at least 40% of the hydroxyl component, such as at least 50% of the hydroxyl component, e.g. at least 60% of the hydroxyl component, such as at least 70% of the hydroxyl component, e.g. at least 80% of the hydroxyl component and at most 20% of the aqueous base, such as at least 90% of the hydroxyl component and at most 10% of the aqueous base, or a stepwise combination thereof.

Since the fraction enriched in polyphenols may comprise many different polyphenolic and chlorogenic acid compounds a stepwise elution may be used. Thus, in an embodiment of the present invention the desorption eluent may comprise
(i) the hydroxyl component in the range 10-30%, such as 15-25%, such as 17-23% or such as 19-21%, or
(ii) the hydroxyl component in the range 40-60%, such as 45-55%, such as 47-53% or such as 49-51%, or
(iii) the hydroxyl component in the range 70-90%, such as 75-85%, such as 77-83% or such as 79-81%.

In a further embodiment of the present invention the desorption step may be a stepwise elution comprising the combination of two or more of the concentrations of hydroxyl components in the desorption eluent as mentioned in (i), (ii) or (iii), such as a stepwise combination of all three. It may also be advantageous to elute by adding a gradient of the concentration of the hydroxyl component during desorption, thereby obtaining an desorption eluent comprising different polyphenolic compounds. Thus, in an embodiment of the present invention the hydroxyl component may be provided as a gradient going from at the most 100% to minimum 1% during desorption, such as from at the most 90% to minimum 15%, such as from at the most 90% to minimum 70%, such as from at the most 60% to minimum 40%, such as from at the most 30% to minimum 10%. In the above embodiment the hydroxyl component is initially a high concentration, however, it may be advantageous to go from a low concentration to a highconcentration. Thus, in another embodiment of the present invention the hydroxyl component may be provided as a gradient going from at least 1% to at the most 100% during desorption, such as from at least 15% to at the most 90% such as from at least 10% to at the most 30%, such as from at least 40% to at the most 60%, such as from at least 70% to at the most 90%. In a further embodiment the desorption step is provided as a stepwise combination of any of the gradient ranges according to the invention. By having a stepwise elution where multiple eluates are collected (each with a different content of the different polyphenols,) it may subsequently be possible to mix two or more of the collected eluates to obtain a final product with an isomeric balance of polyphenols, in particular chlorogenic acids,which resembles the isomeric balance of polyphenols, in particular chlorogenic acid, present in the starting material more accurately. If only one eluate is collected, this may not be possible.

As mentioned previously, temperature influences the formation of ethylesters. Thus, in an embodiment of the present invention desorption may be performed at a temperature below 80° C., such as below 75° C., such as below 50° C., such as below 35° C., or such as in the range 20-30° C. However, since the speed of the desorption is faster at higher temperature, this is again a tradeoff

After desorption it may be an advantage to further adjust the pH of the eluate to avoid ethylester formations. Thus, in an embodiment the pH is adjusted after desorption to a pH in the range 3.5-6, such as 3.5-5, such as 3.5-4.5, such as 4-5, or such as around 4 or 5.

The eluate obtained comprises a hydroxyl component and in order to further enhance the concentration of the polyphenols in the eluate the hydroxyl component may be separated from the polyphenols.

The separation step after elution, and optional pH adjustment, may be performed in different ways. In an embodiment of the present invention the separation in step (vi) may performed by evaporation. Since temperature also influences ethylester formation the evaporation of the eluate is performed at a temperature below 80° C., such as below 75° C., below 60° C., such as in the range 40-60° C., or such as in the range 45-5° C., preferably around 50° C. However, since the speed of the desorption is much faster at elevated temperatures, this is again a tradeoff. After evaporation an evaporated phase comprising the hydroxyl component and a condensate phase comprising the polyphenols are provided.

Preferably the content of hydroxyl component present in the residual phase is less than 5% (w/w), more preferably less than 1% (w/w), even more preferably less than 0.1% (w/w), even more preferably less than 0.01% (w/w).

It is to be understood that pH adjustments may take place before separation (such as evaporation) of the hydroxyl component or after separation (such as evaporation) of the hydroxyl component. In addition, pH adjustment may take place both before and after separation of the hydroxyl component. Thus, in an embodiment of the present invention the pH of the desorption eluent may be adjusted to a pH in the range 3.5-6, such as 3.5-5, such as 3.5-4.5, such as 4-5, or such as around 4 or 5. In an embodiment of the present invention the separation step, such as evaporation takes place before separation, after separation or both before and after separation. After a separation step the desorption eluent may be present in an acidic solution in which the polyphenols may be present in their hydrophobic and predominantly insoluble form. In order to better solubilise polyphenols, a pH adjustment as described above may be an advantage.

In order to make the purification protocol industrial applicable, processing time may be important. Thus, in an embodiment of the present invention the processing time may be 36 hours or less, such as 30 hours or less, e.g. 25 hours or less, such as 20 hours or less, e.g. 18 hours or less, such as 15 hours or less, e.g. 12 hours or less, such as 10 hours or less, e.g. 5 hours or less. In the present context the processing time relates to the period from solubilization of the starting material until the eluate has been separated into a phase comprising the hydroxyl component and a residual phase comprising the polyphenols thereby obtaining the fraction enriched in polyphenols according to the invention.

The fraction enriched in polyphenols may be further purified. Thus, in an embodiment of the present invention the fraction enriched in polyphenols may be further purified by chromatography, ultrafiltration, nanofiltration, microfiltration, reversed osmosis or any combination thereof. The final product may also be freeze-dried.

Besides relating to a purification process, the present invention also relates to products obtainable by such process. Thus, another aspect of the present invention relates to a fraction enriched in polyphenols obtainable from the process according to the present invention. Such products may be very unique as they comprise a balance of polyphenols, in particular the balance of cholorogenic acids, that resembles the balance found originally in the starting material.

In an embodiment of the present invention the fraction enriched in polyphenols may have a content of polyphenols of at least 35% (w/w) on a dry matter basis, such as at least 45%, such as at least 55%, such as at least 65%, or such as at least 75%. Thus, in an embodiment of the present invention the fraction enriched in polyphenols may have a content of ethylester of polyphenols below 20% (w/w) on a drymatter basis and relative to the total content of polyphenols, such as below 15% (w/w), e.g. below 10% (w/w), such as below 5% (w/w), e.g. below 1 (w/w), such as in the range of 0.1-20% (w/w), such as in the range 0.1-10%, such as in the range 0.1-5%, such as in the range 0.1-4%, such as in the range 0.1-3%, such as in the range 0.1-2%, or such as in the range 0.1-1%. In a more specific embodiment of the present invention the fraction enriched in polyphenols may have a content of polyphenols of at least 35% (w/w) on a dry matter basis and below 20% (w/w) on a drymatter basis and relative to the total content of polyphenols, such as between 0.1-20% (w/w) ethylesters of polyphenols.

Thus, in an embodiment of the present invention the polyphenol may be a chlorogenic acid (CGA), caffeic acid or a combination thereof. In yet an embodiment of the present invention the chlorogenic acid (CGA) may be selected from the group consisting of 3-caffeoyl quinic acid (3CQA), 4-caffeoyl quinic acid (4CQA), 5-caffeoyl quinic acid (5CQA), 3,4-dicaffeoyl-quinic acid (3,4diCQA), 3,5-dicaffeoyl-quinic acid (3,5diCQA), 4,5-dicaffeoyl-quinic acid (4,5diCQA), 3-feruloyl quinic acid (3FQA), 4-feruloyl quinic acid (4FQA), 5-feruloyl quinic acid (SFQA), caffeic acid and any combination thereof.

In yet another embodiment of the present invention the fraction enriched in polyphenols may comprise 3-caffeoyl quinic acid (3CQA), 4-caffeoyl quinic acid (4CQA), 5-caffeoyl quinic acid (5CQA), 3,4-dicaffeoyl-quinic acid (3,4diCQA), 3,5-dicaffeoyl-quinic acid 3,5diCQA), 4,5-dicaffeoyl-quinic acid (4,5diCQA), 3-feruloyl quinic acid (3FQA), 4-feruloyl quinic acid (4FQA), 5-feruloyl quinic acid (SFQA) and caffeic acid.

In yet an embodiment of the present invention the enriched fraction has a content of each of 3-caffeoyl quinic acid (3CQA), 4-caffeoyl quinic acid (4CQA), 5-caffeoyl quinic acid (5CQA), 3,4-dicaffeoyl-quinic acid (3,4diCQA), 3,5-dicaffeoyl-quinic acid (3,5diCQA), 15 4,5-dicaffeoyl-quinic acid (4,5diCQA), 3-feruloyl quinic acid (3FQA), 4-feruloyl quinic acid (4FQA) and 5-feruloyl quinic acid (SFQA) that deviates at most by 30% (w/w) relative to the content present in the starting material, such as at most 25% (w/w); e.g. at most 20% (w/w), such as at most 15% (w/w); e.g. at most 10% (w/w).

In another embodiment of the present invention the chlorogenic acid (CGA) may comprise at least 50% (w/w) mono-caffeoyl quinic acid and/or at most 25% (w/w) di-caffeoyl quinic acid. In another similar embodiment the fraction enriched in polyphenols comprises at least 12% (w/w) on a dry matter basis of at least one of each of 3-caffeoyl quinic acid (3CQA), 4-caffeoyl quinic acid (4CQA) or 5-caffeoyl quinic acid (5CQA), such as at least two of said compounds, preferably all three of said compounds.

In yet an embodiment of the present invention the fraction enriched in polyphenols may comprise less than 12% (w/w) on a dry matter basis of at least one of each of 3,4-dicaffeoyl-quinic acid (3,4diCQA), 3,5-dicaffeoyl-quinic acid (3,5diCQA) or 4,5-dicaffeoyl-quinic acid (4,5diCQA) such as at least two of said compounds, preferably all three of said compounds.

In yet an even further embodiment of the present invention the fraction enriched in polyphenols may comprise less than 12% (w/w) on a dry matter basis of at least one of each of 3-feruloyl quinic acid (3FQA), 4-feruloyl quinic acid (4FQA) or 5-feruloyl quinic acid (5FQA), preferably all three.

The fraction enriched in polyphenols may be used directly as a food ingredient or form part of a food ingredient. Thus, a further aspect of the present invention relates to a food ingredient comprising the fraction enriched in polyphenols according to the present invention. In an embodiment of the present invention the food ingredient may comprise between 0.1-20% (w/w) ethylesters of polyphenols relative to the total amount of the polyphenols in the food ingredient, such as in the range 0.1-10%, such as in the range 0.1-5%, such as in the range 0.1-4%, such as in the range 0.1-3%, such as in the range 0.1-2%, or such as in the range 0.1-1%.

The fraction enriched in polyphenols or the food ingredient may be used in a food product. Thus, yet an aspect of the present invention relates to a food product comprising the fraction enriched in polyphenols and/or the food ingredient according to the present invention. In an embodiment the food product comprises between 0.1-20% (w/w) ethylesters of polyphenols relative to the total amount of the polyphenols in the food product, such as in the range 0.1-10%, such as in the range 0.1-5%, such as in the range 0.1-4%, such as in the range 0.1-3%, such as in the range 0.1-2%, or such as in the range 0.1-1%.

Polyphenols may be used for different purposes to improve a food product. Thus, an aspect of the present invention relates to the use of the fraction enriched in polyphenols according to the present invention and/or the food ingredient according to the present invention as a precursor for generating aroma, flavor and/or foaming.

In a further aspect the invention relates to a pharmaceutical composition comprising the fraction enriched in polyphenols according to the present invention and a pharmaceutical acceptable carrier. Since polyphenols may exhibit biological activity in vivo e.g. antioxidants, activator/inhibitor of enzyme and receptors they can be considered relevant components in pharmaceutical compositions.

It should be noted that embodiments and features described in the context of one of the aspects of the present invention also apply to the other aspects of the invention.

The invention will now be described in further details in the following non-limiting examples.

Methods

Fractions

Green coffee fractions were manufactured from either plain or decaffeinated green Robusta beans.

Resin

The Amberlite FPX66 (CAS N° 9003-69-4) is a commercial food grade resin which chemical structure consists of a macro reticular aromatic polymer, namely styrene. As the styrene rings are not functionalised, the matrix is rather hydrophobic.

Purification Setup for Labscale Experiments
Columns: XK 50/30 glass columns with thermo jacket (GE Healthcare)

Purification Set-Up for Bench Scale
Columns: XK 50/100 glass columns with thermo jacket (GE healthcare)

Chlorogenic Acids Analysis by HPLC-UV

Chlorogenic acid (CGA) analysis was performed by HPLC-UV with the following set up:
System—HPLC is an Ultimate 3000, supplied by Dionex®. It is equipped with a quaternary pump, a sample injector, a UV detector and Chromeleon software.
Sample Preparation—The samples were powders or fractions. Powders were solubilized in methanol:water (80:20) at a concentration of 2 mg/mL. Fractions were diluted in methanol:water (80:20) so that the analyte ranged below 0.2 mg/mL.
HPLC Method—The chromatographic separation was achieved on a Spherisorb ODS-1 column, 5 μm, 250×4.6 mm (Waters) at ambient temperature. A binary gradient of A/B (A: 92% water/8% acetonitrile/1 mL/L ortho-phosphoric acid, B: 50% water/50% acetonitrile/2 mL/L ortho-phosphoric acid) was applied. The injection volume was 20 μL. The wavelength was set at 325 nm for chlorogenic acids and 275 nm for caffeine analysis.
Chlorogenic acid isomers—The used method enabled to determine concentrations for caffeine and the nine major chlorogenic acids: the 3 caffeoyl quinic acids (CQA), the 3 feruloyl quinic acids (FQA) and the 3 di-caffeoyl quinic acids (di-CQA), in 45 minutes
Calibration—The calibration is performed with external standards. Two standards are used (5-CQA and caffeine) at a concentration of 0.2 mg/mL in methanol:water (80:20). Conversion factors of chlorogenic acid isomers were determined using pure standards from Biopurify.

Concentrations, expressed in gram per 100 gram, of the other CQA are determined from that of the 5-CQA thanks to this formula:

$$W = \frac{{A}_{s} \times {C}_{0} \times P \times {V}_{s} \times D \times F \times 100}{{A}_{0} \times {M}_{s} \times 1000}$$

Where:
W is the analyte concentration in gram per 100 grams of fresh product.
Aₛ is the peak area of analyte in the test sample solution.
C₀ is the concentration of analyte in the standard in milligram per millilitre (5-CQA if analyte is not in the standard solution).
P is the purity of the analyte in the standard when purity is less than 0.95 (If purity ≧0.95, P=1).
Vₛ is the volume of the test solution in millilitre.
D is the dilution factor (10 or 1).
F is the correction factor allowing the calculation of other chlorogenic acid isomers from the 5-CQA standard.

LC-MS Analysis for Desorption Study and Fraction Composition

The coffee fractions were characterized by LC-UV-MS/MS as follows:
System: The analysis was performed using an 1200SL, a DAD detector (Agilent) and a QToF 6520 (Agilent)
Sample Preparation: Direct injection
HPLC Method: The chromatographic separation was achieved on a Kinetex C18 column, 2.6 μm, 100×2.1 mm (Phenomenex) coupled to a QToF 6520 (Agilent). A binary gradient of A/B (A: 0.1% formic acid, B: 0.1% formic acid in MeOH) was applied.
Column temperature was kept at 30° C. The sample concentration was approximately 10 mg/mL in water. The injection volume was 3 μL. The wavelength was set at 325 nm, the reference was settled at 385 nm and for both the band width was 2 nm.
MS parameters: The mass spectrometer QTOF 6520 (Agilent) was used in positive and negative ESI-mode and was operated in MS mode to follow the desorption of chlorogenic acids and auto MS/MS mode for compound identification.

### EXAMPLE 1

### Precipitation

Green coffee extracts (i.e. caffeinated/decaffeinated) were prepared at 50% TC (dry matter content) in boiling water without any problems.

As untreated extract can lead to clogging of the column, the high molecular weight (HMW) compounds (i.e. proteins, melanoidins, arabinogalactans) of green coffee extract were eliminated by precipitating them in 80% ethanol (V/V). The precipitate was removed by filtration on a Buchner filter (porosity grade 1). Ethanol was further evaporated. Afterwards the concentration of CGAs was evaluated by HPLC-UV before and after treatment. The main results were:
No clogging of the column was observed after precipitation of HMW compounds.
An 80% ethanol treatment did not affect chlorogenic acid solubility.
Losses in CGA were low and mostly embedded in the precipitate.

In conclusion alcohols, such as ethanol, are particular useful in the process according to the invention.

### EXAMPLE 2

### Adsorption

The adsorption was first assessed with a coffee solution as obtained from example 1 as such. This revealed a poor adsorption rate. Next the coffee solution, as obtained from example 1, was acidified to pH 1 to reinforce the strength of the hydrophobic interactions between the CGA species and the aromatic polymer of the resin. Analysis of CGAs by HPLC-UV revealed that:
The extract without acidification was poorly retained (11%).
Acidification to pH 1 significantly improved the retention of polyphenols. The adsorption rate was nearly doubled with the acidification step as compared to natural pH of the coffee extract i.e. pH 4-7 The final adsorption rate was increased to about 19%. That value was later further improved by optimisation of the ratio coffee/resin.
Under acidic conditions only CGA and other hydrophobic compounds are retained by the column whereas hydrophilic molecules (e.g. minerals and sugars) pass through.

In conclusion an acidic environment improves the adsorption step of the present disclosed protocol.

### EXAMPLE 3

### Column Rinsing

To eliminate the compounds that were not retained but still present in the dead volume of the column, a rinse with water and 0.1M HCl was carried out. Wash water was checked for CGAs afterwards. As the main findings it was found that:
Pure water tends to desorb CGAs.
Under acidic condition, as chlorogenic acids remain in their protonated and hydrophobic form, interactions with the resin were maintained. CGA were not desorbed upon column rinsing with 0.1M HCl.
Purity of the final enriched fraction increases from 50% to 66% when including the washing step.

In conclusion a washing step increases the purity of the final product.

### EXAMPLE 4

### Desorption

For CGAs desorption, the hydrophobic interactions between the resin and phenolic compounds had to be weakened. With a view to a food grade purified fraction, ethanol was chosen as organic solvent. KOH (10⁻⁴ M) was added in order to increase the pH of the desorption solution. This ethanol/KOH mixture (90/10 V/V) was used as desorption solvent and compared to pure ethanol or ethanol/water mixture. The findings were:
When using ethanol/water, CGA are not fully desorbed.
Desorption efficiency can be improved significantly by increasing the pH of the desorbing medium to 5. By the use of ethanol/KOH CGA are partially de-protonated, i.e. they have less affinity for the resin and therefore 100% desorption is observed.
Increasing the temperature to 70° C. reduces desorption duration by 30%.

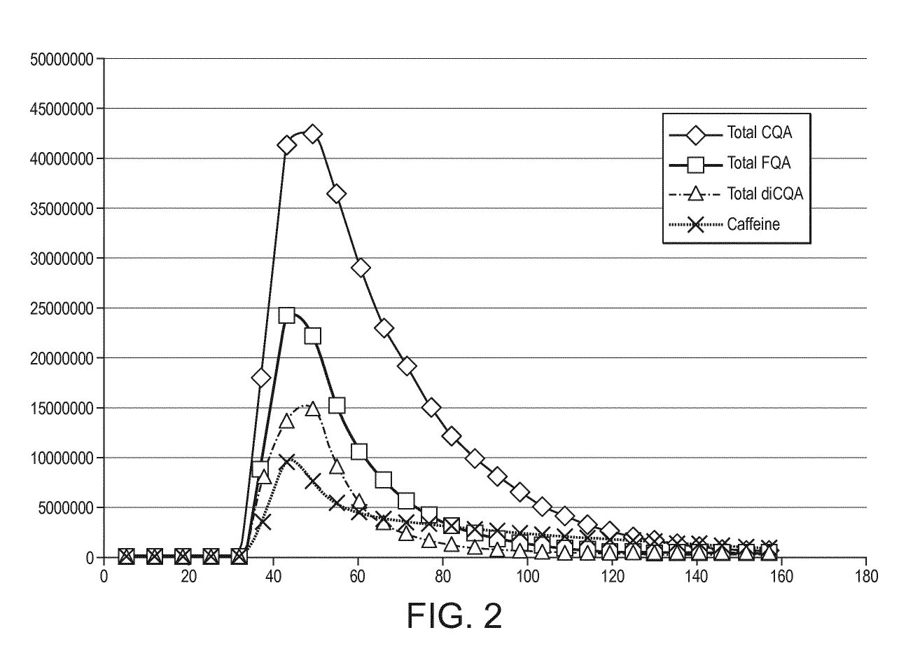

To ensure that the composition of CGA in the purified fraction remains the same as in the initial coffee it is crucial to know the desorption kinetics of the different isomers and for caffeinated samples that of caffeine as well. Therefore a series of fractions was collected upon desorption. As displayed in FIG. 2 this study revealed:
With 90% ethanol CQAs, FQAs and Di-CQAs are eluted within the same time interval allowing desorption of all isomers at once.
For the single isomers the maximum of desorption is also within a small time range (data not shown).
Caffeine starts eluting with the CGA but is desorbed more slowly than CGA.

### EXAMPLE 5

### Coffee/Resin Ratio

To optimise the coffee/resin ratio, fraction collection at the output of the column during the adsorption phase was performed. Each fraction was analyzed by UV at 325 nm (characteristic wavelength of CGA). The optimal ratio corresponds to the volume of coffee solution at which CGA begin to elute again out of the column. From this volume of solution, the corresponding mass of coffee solids that pass through the column was calculated. The optimal coffee/resin ratio was determined to be approximately 0.55 g of coffee for 1 g of resin. However, as the content of chlorogenic acid may vary considerably from one type of coffee to another and within the same sort of coffee this ratio may change.

### EXAMPLE 6

### Composition Before and After Purification—Labscale

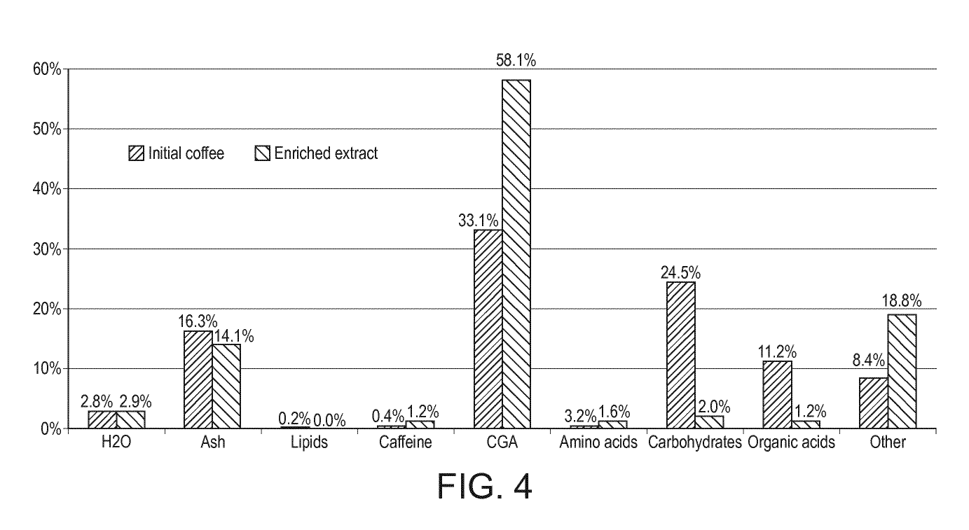

After having completed the purification protocol according to FIG. 3, the components were compared to the content of the starting material. The freeze-dried powder was analyzed for its composition in order to evaluate the changes of other compounds that occur during purification of CGAs. As displayed in FIG. 4 the major changes are the following:
The major change concern CGAs. Their level is about 2 times higher in the enriched purified fraction;
Carbohydrates and organic acids significantly decrease (amounts divided by 12 and 10, respectively) due to their high polarity;
Amino acids are reduced as well but less than carbohydrates and organic acids;
Caffeine is present and undergoes enrichment up to 1.2% (3 times more) although the initial coffee is a decaffeinated one;
The proportion of compounds not identified, listed in the “other” category, is multiplied by 2.2. The compounds responsible for this increase might be of same hydrophobicity than CGA and are therefore also adsorbed during the process.

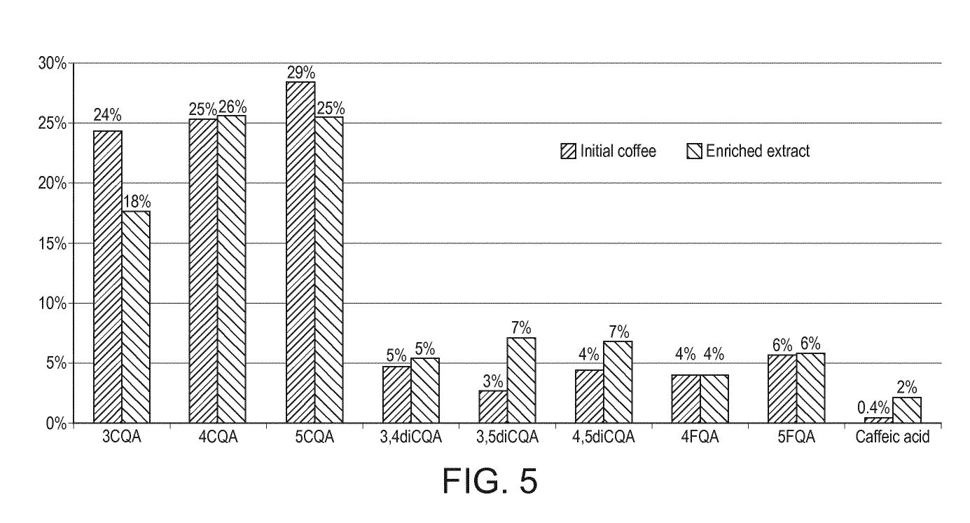

The detailed composition of the enriched polyphenol fraction was further evaluated. Analysis of the CGA profile revealed the following results as shown in FIG. 5.
The CGA level increases from 33% to 56% under the labscale purification protocol. The initial balance in CGA isomers is rather maintained in the enriched purified fraction;
The FQA balance remains the same.
The relative content in CQA is slightly reduced, especially that of 3-CQA. The higher polarity of this isomer can explain its poorer adsorption on the resin.
The content in di-CQA, mainly 3,5 and 4,5-di-CQA, increases. The two caffeic acid residues may confer a higher affinity of these isomers for the resin (two interactions instead of one for FQA and CQA), enabling a more efficient purification.
The proportion in free caffeic acid significantly increases (amount multiplied by 5), possibly due to hydrolysis of CQAs during the purification process.

In total the isomer balance can be considered as close to that of the initial extract.

After successive optimization of all purification steps a CGA fraction with a purity of ˜70% and preserved CGA balance could be obtained. The enrichment factor was globally around 2. The key steps of the process are:
The pre-purification by precipitation of the high molecular weight compounds in ethanol 80%.
The adsorption phase on the hydrophobic resin (i.e. Amberlite FPX66), optimized by acidification of the extract at pH 1 prior to absorption.
The desorption phase carried out with ethanol/KOH 10⁻⁴M (90/10) at 70° C.
The pH adjustment at 5 to maintain the solubility of CGA during evaporation and improve the performance of the fraction upon freeze-drying.
The enriched fraction was also enriched similarly in other hydrophobic compounds containing aromatic moiety such as minor CGA, NPPA, DKP, and caffeine.

### EXAMPLE 7

### Pure Soluble Coffee Composition Before and After Purification—Lab Scale

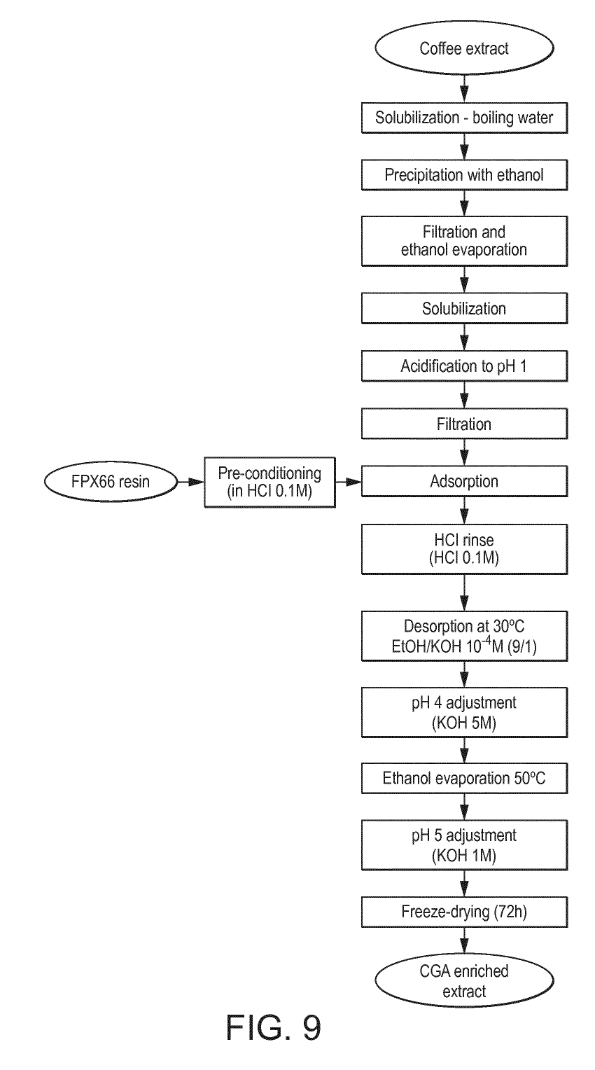

From the findings of the labscale study in example 6, the optimized protocol as shown in FIG. 9 was applied for Pure Soluble Coffee. In the present example the following conditions were applied:
Pure soluble coffee was used as starting material
Polyphenol enriched fraction production was carried out with lab-scale column.

The results are:
For the enriched fractioned CGA content is increased from 10% to 24% under the labscale purification protocol. The initial balance in CGA isomers is rather maintained in the enriched purified fraction;
The CGA level increases from 33% to 56% under the labscale purification protocol. The initial balance in CGA isomers is rather maintained in the enriched purified fraction;
The optimized protocol can be applied for Pure Soluble Coffee

### EXAMPLE 8

### Avoiding Formation of Ethylester from CGAs

The process conditions favoring the undesirable formation of ethylester from CGA were investigated. The kinetics of the degradation of CGA and respectively the formation of CGA esters were followed in model studies. During the process ethylesters of CGA may be formed. This is however, undesirable because it may significantly influence the CGA balance and the properties of the enriched fraction.

The scheme below displays the general reaction mechanism of an esterifaction resulting in the ethylester. The reaction between an acid and an alcohol is catalyzed by proton transfer making the reaction rate pH-dependent. As a second key parameter the temperature should influence the degradation rate as it is usually the case for chemical reactions. Both parameters were evaluated in order to determine the ideal conditions to avoid an ethylation of CGAs.

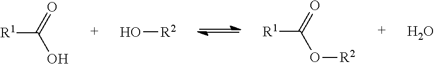

During the experiment 5-CQA was stored in the presence of ethanol (75% ethanolic solution) by varying the following parameters pH, temperature and time.

The process was conducted under the following conditions using 5-CQA esterification as the model.

For each trial 10 mg of 5-CQA were dissolved in 40 mL of a 75% ethanolic solution.
pH studies: The pH was adjusted to pH 1, pH 2.7 and pH 4 with hydrochloric acid (32%) and to pH 12 by using NaOH solution (4 N) and all samples were kept in a lab oven at a temperature of 80° C. for 2 hours.
Temperature studies: CQA solutions were adjusted to pH 1 with hydrochloric acid (32%) and kept for 2 hours either at room temperature, 50° C. or 80° C. in a lab oven.
Time studies: 5-CQA solutions were adjusted to pH 1 and kept at room temperature or 50° C. for 1 h, 2 h, 3 h and 36 h (RT only).
System: The analysis was performed on a LC-DAD-MS system using LCQ Deca mass spectrometer (Thermo Finnigan), with a DAD detector (Thermo Finnigan) and a Rheos HPLC system (Thermo Finnigan).
Sample Preparation: For each parameter an aliquot of the solution was diluted 1:10 and directly used for LC/MS-DAD analysis.
HPLC Method: The separation was performed at ambient temperature on a Zorbax Eclipse C18 column, 5 μm, 150*3 mm (Agilent). A binary gradient of A/B (A: 0.1% formic acid, B: methanol) was applied.
MS parameters: The Mass spectrometer used is the LCQ-Deca operated in FullScan and data dependant MS/MS mode (m/z 100-1000) for compound identification.

pH

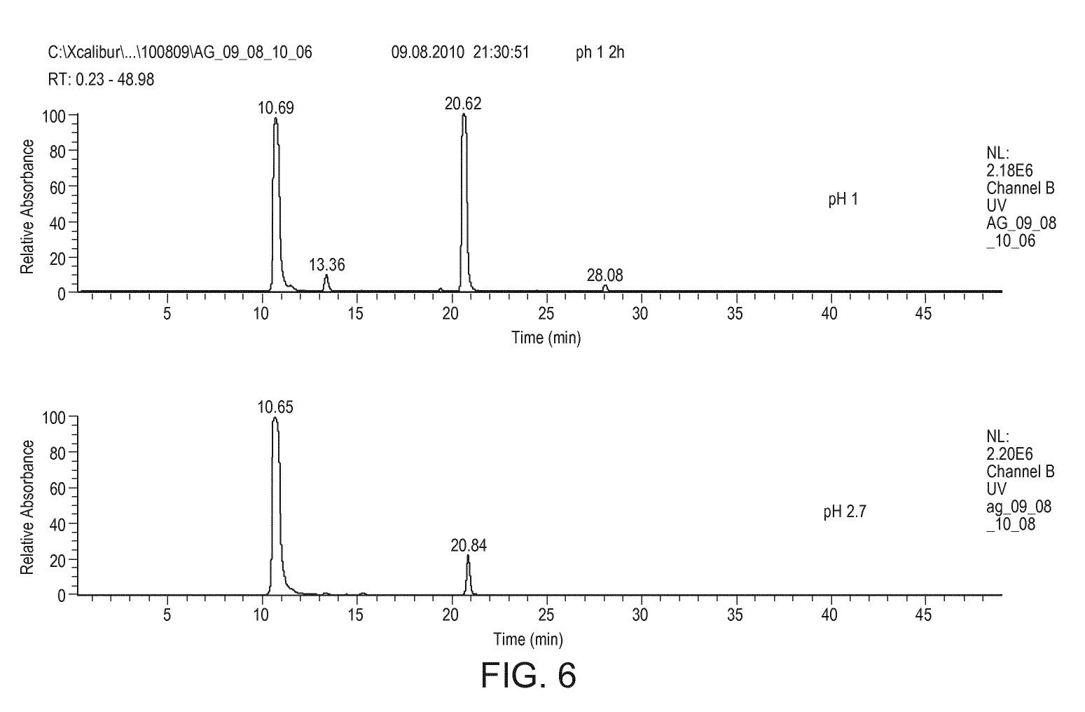

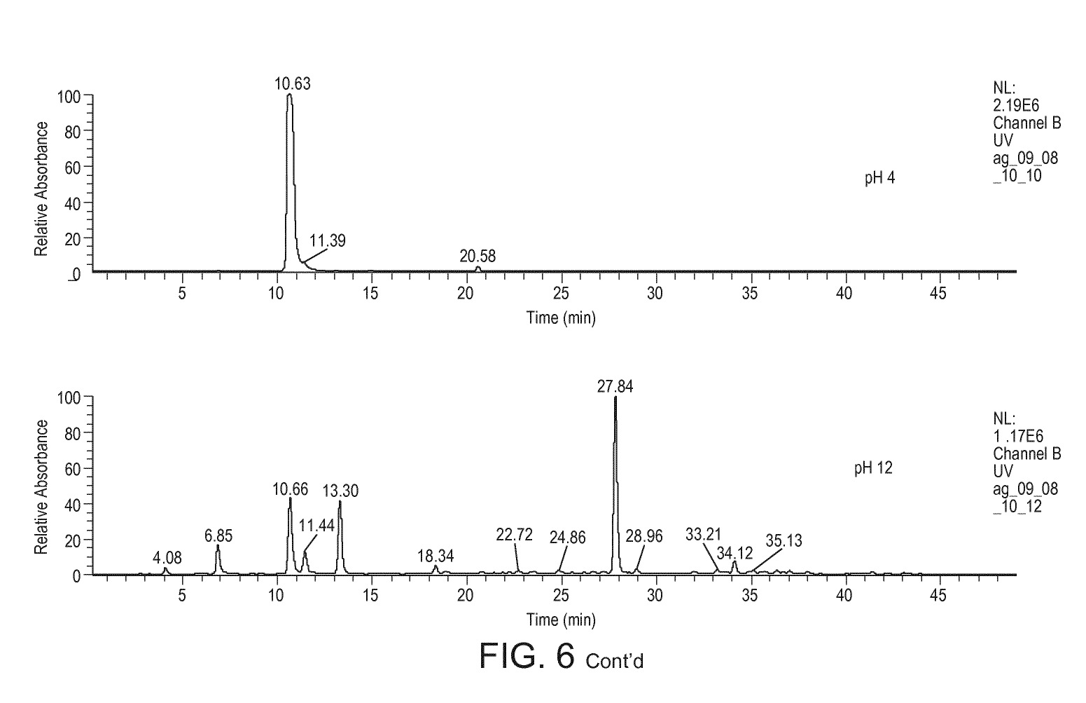

The degradation of 5-CQA and the formation of the corresponding ethyl-ester were monitored by LC/MS-DAD. FIG. 6 shows the chromatograms of 5-CQA stored for 2 h at different pH in a 75% ethanol solution. The study on pH shows:
At pH 1, a rapid transformation of 5-CQA into its ester is observed within 2 h.
At pH 2.7, 5-CQA esterification is significantly reduced.
At pH 4, 5-CQA is almost not degraded after 2 h.
Strong alkaline conditions (pH 12) induce a complete different reaction scheme including hydrolysis of 5-CQA yielding caffeic and quinic acid. In addition alkaline conditions favor the isomerisation of 5-CQA as observed by the appearance of 3CQA and 4-CQA.

In conclusion, in presence of ethanol the pH of the enriched fraction should be around 4, such as 4-5. Thus, a pH around 4-5 may be optimal for step (v) in the process according to the present invention as displayed in FIG. 6, wherein it can be seen that at lower pH's (pH 2.7 and below) ethylester formation takes place, whereas at higher pH's (pH 12) hydrolysis of 5-CQA yielding caffeic and quinic acid takes place. In addition alkaline conditions favor the isomerisation of 5-CQA as observed by the appearance of 3-CQA and 4-CQA.

Temperature

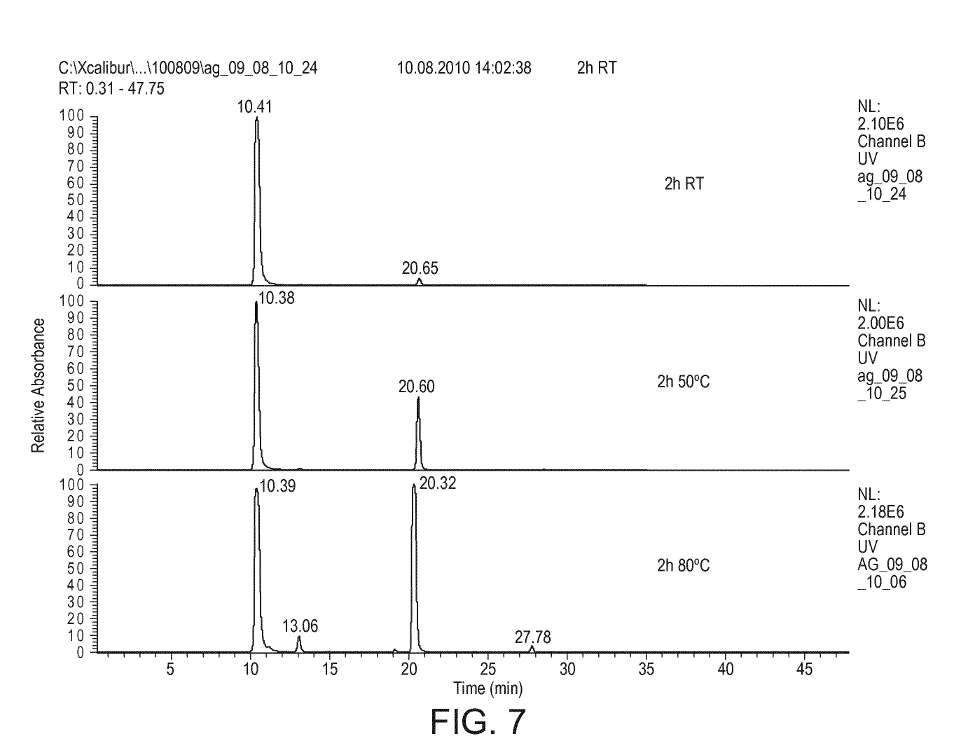

In a next step, the influence of the temperature was evaluated. To simulate the worst case scenario, pH 1 was chosen. Indeed, it corresponded to the most critical pH in the previous experiment and it currently reflects the conditions of the purification protocol. After 2 h the degradation of 5-CQA kept at three temperatures, namely ambient (RT), 50° C. and 80° C., was monitored by LC/MS-DAD (FIG. 7). The studies on temperature show:
At RT only a small amount of 5-CQA is transformed into its ethylester.
At 50° C. the esterification rate increases. Up to 40% of the ester are detectable as compared to the native 5-CQA.
At 80° C. the esterification of 5-CQA into its ethylester is further accelerated resulting in same amounts of the ester as compared to 5-CQA.

In conclusion, in presence of ethanol, the temperature of the coffee extract and the enriched fraction should be lowered, in particular under acidic conditions.

Time

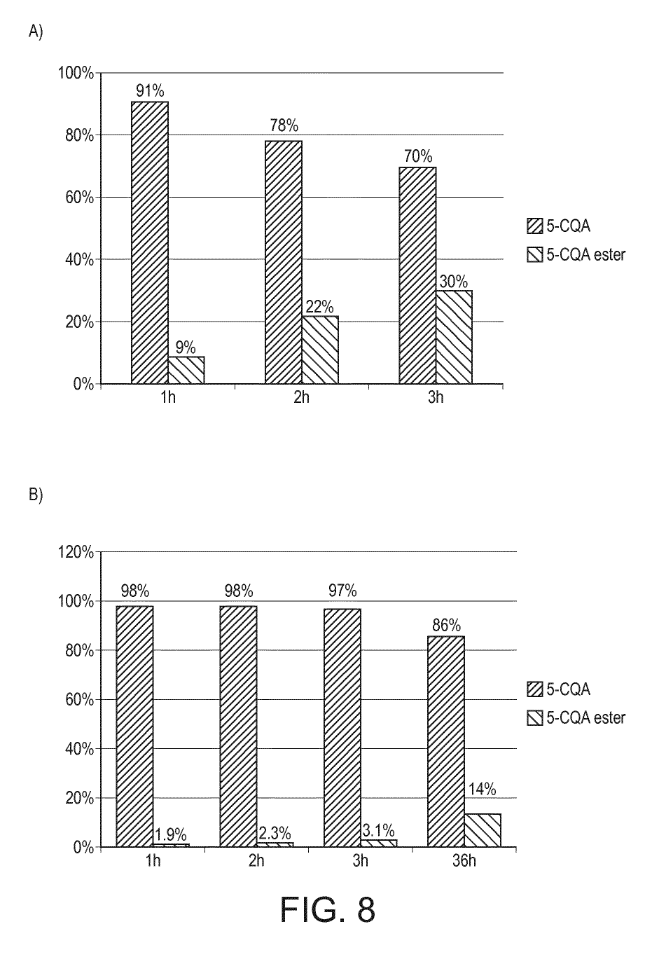

While pH and temperature are independent from the size of the purification setup, the residence time is clearly increased with up-scaling. Therefore the next study evaluated the esterification kinetics of 5-CQA into its ethylester at given pH and temperature. This study was performed at pH 1 for both room temperature and 50° C. (FIG. 8). The studies on time show:
At 50° 5-CQA is converted steadily into its ethylester within the first 3 h. 30% of the ester are formed after a period corresponding to the current desorption duration of the up-scaled protocol.
At room temperature the stability of 5-CQA is much higher. Only 3% of the ester is formed after 3 h. It increases up to 14% after 36 h.

In conclusion, the formation of ethylesters depends on pH and temperature of the desorption step.

### EXAMPLE 9

### Decaffeinated Coffee Composition Before and After Purification—Large Scale

From the findings of the labscale study in example 6, the optimized protocol (FIG. 9) was upscaled for larger production of enriched fractions without degradation of CGA. In the present example the following conditions were applied:
Decaffeinated green coffee extract was used as starting material
Polyphenol enriched fraction production was carried out with the 1 m-column.

5 batches were produced in order to assess the repeatability of the purification process.

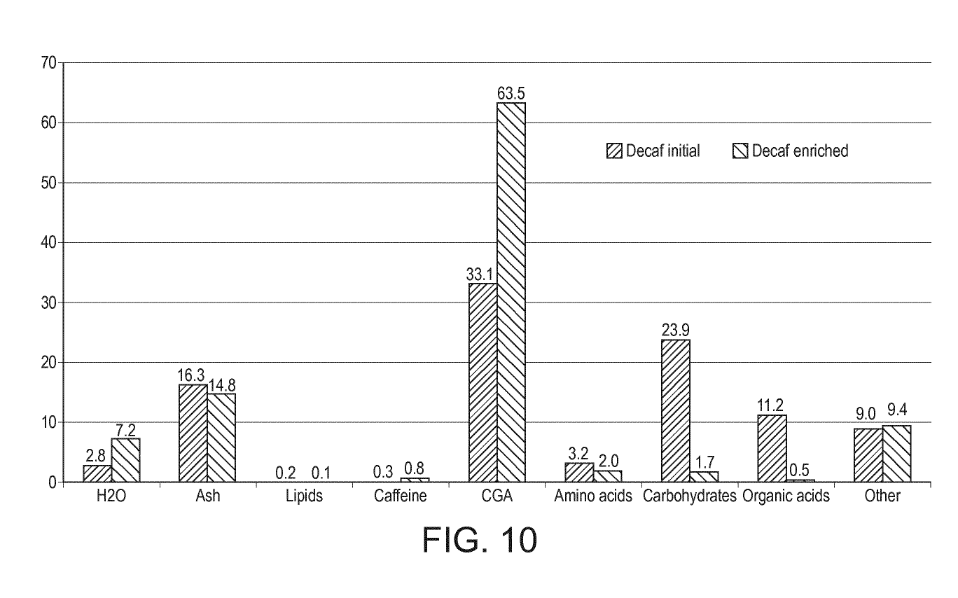

A complete characterization of the decaffeinated fraction was performed and the results are displayed in FIG. 10.
For the decaffeinated enriched fractioned CGA content is increased from 33% to 63%.
The changes in composition upon purification are similar to those observed during lab-scale preparation (see example 6).
As compared to lab scale preparation the group of other compounds is less enriched (9.4% vs 18.8%).
The enrichment of caffeine is lower as compared to labscale (0.8% vs 1.2%).

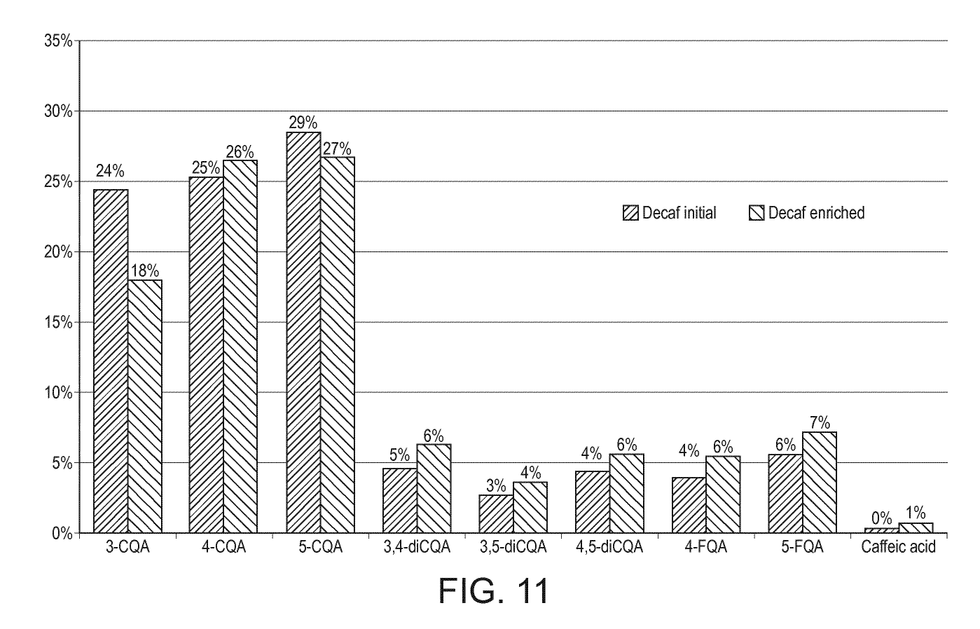

The detailed composition of CGA is displayed in FIG. 11.

As already seen under lab scale conditions the CGA balance is maintained quite well (Example 6).
The major change concerns 3-CQA as the most polar compound among CGA that is less abundant after purification.
In contrast more apolar di-CQA and FQA are enriched upon purification.

Moreover, the updated protocol (FIG. 9) was evaluated for its performance.
The overall recovery was 24%.
The recovery of CGA was 46%.
CGA were enriched by a factor of 1.9 as compared to the initial amount.

In addition, the analysis of CGA and of their esters in the final product with HPLC-UV/MS revealed:
The transformation of ethylester was drastically reduced
The final concentration of esters did not exceed 1% of the native CGA

Thus, it is indeed possible to perform the process according to the present invention for decaffeinated compositions at a scale which is industrially applicable, without drastically affecting the polyphenols balance or generating large amounts of ethylesters.

### EXAMPLE 10

### Caffeinated Coffee Composition Before and After Purification—Large Scale

For the caffeinated polyphenol enriched fraction, the updated protocol (FIG. 9) was also applied with the following conditions:
Caffeinated green coffee extract was used as starting material
Polyphenol enriched fraction production was carried out with two 60 cm columns used in parallel to reduce residence time.
batches were produced in order to assess the repeatability of the purification process.

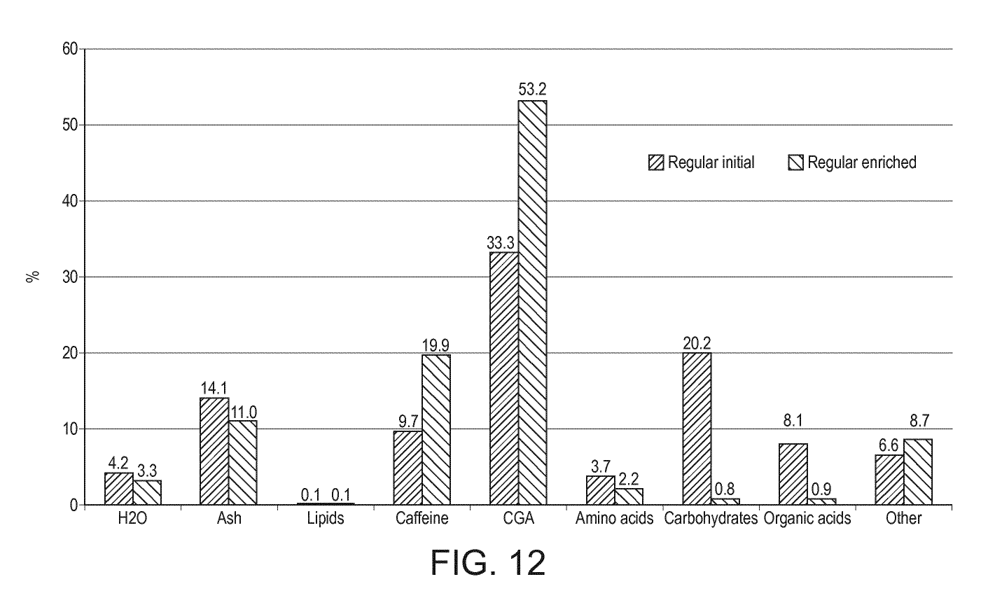

A complete characterization of the caffeinated fraction was performed and the results are available in FIG. 12.
For the caffeinated enriched fractioned CGA content is increased from 33% to 53%.
The amount of caffeine is more than doubled from 9.7% to 19.9%.
Carbohydrates and organic acids are drastically reduced due to their high polarity.
Other compounds are slightly increased.
The changes are similar as those observed for decaffeinated enriched fraction.

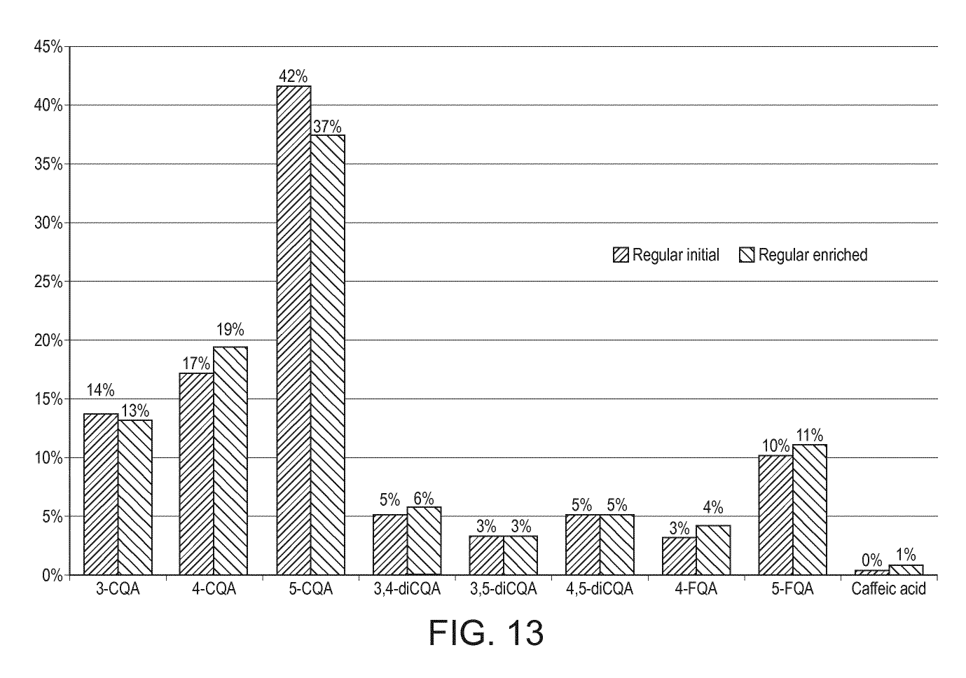

The detailed composition of CGA in the enriched caffeinated fraction is displayed in FIG. 13.
In contrast to the decaffeinated enriched fraction the 3-CQA concentration remains almost unchanged.
The major change concerns the 5-CQA that is less abundant after purification.
Overall the changes in composition are less evident then for the decaffeinated enriched fraction and the profile remains very similar to that of the initial coffee.

For the caffeinated enriched fraction ten batches were analysed separately to evaluate the batch-to-batch performance.
The overall recovery was found to be 34% and was therefore higher as for the decaffeinated enriched fraction (24%).
The recovery of CGA was at 55% as compared to 46% for the decaffeinated enriched fraction.
CGA were enriched by a factor of 1.6 as compared to 1.9 for the decaffeinated one.

An HPLC-UV/MS was also performed to control the content on CGA and their esters in the enriched caffeinated fraction. Data not shown.
The final concentration of esters did not exceed 1% of the native CGA.
As already seen for the decaffeinated enriched fraction the formation cannot be avoided completely.

Thus, it is indeed possible to perform the process according to the present invention for caffeinated compositions at a scale with is industrially applicable, without drastically affecting the polyphenols balance or generating large amounts of ethylesters.

### EXAMPLE 11

### Comparison of the Performance for the Preparation of Enriched Decaffeinated and Caffeinated Fractions

Conclusions:
A fraction of 63% purity can be obtained with the protocol displayed in FIG. 9 for decaffeinated coffee and 53% for the caffeinated one.
The better performance of decaffeinated fraction might be due to the presence/absence of caffeine. As caffeine is enriched by a factor of 2 it reaches a concentration of almost 20% for the caffeinated fraction which reduces the relative concentration of CGA at the same time.
For other compounds no specific differences between decaffeinated and caffeinated fractions are observed upon purification.
The ratios of 3-CQA, 4-CQA and 5-CQA differ between decaffeinated and caffeinated fractions. This is due to an isomerisation that already occurs during the decaffeination process. The purification process has only little impact on the composition of CGA in the enriched fractions as compared to the initial distribution, thus the isomer balance of polyphenols is maintained throughout the process.
In both cases the concentration of esters does not exceed 1% of the native CGA.
The ester formation cannot be fully avoided without changing completely the protocol due to high amounts of CGA and ethanol present during the process. Amounts of esters of less than 1% must therefore be considered as technically unavoidable for a food grade preparation of CGA enriched fractions.
The reproducibility from batch-to-batch is very good with a relative standard deviation of less than 10%. Therefore the protocol is suitable to deliver from one batch to the other a fraction enriched in CGA with a specified amount of CGA and a well-known balance of single isomers.

### EXAMPLE 12

### Optimization of Ethanol Concentration

A foodgrade protocol to purify coffee polyphenols has been described in the previous examples, with adsorption of coffee polyphenols onto a hydrophobic resin. Desorption was performed under isocratic conditions using 90% aqueous ethanol with a polyphenol purity of 60-70% in the final enriched fraction.

With a view to produce more specific polyphenol products, the preparation of purified coffee polyphenol isomers was investigated. For this purpose, successive desorption steps with solvent of decreasing polarity (i.e. aqueous ethanol 20/80, 50/50, 80/20) were applied.

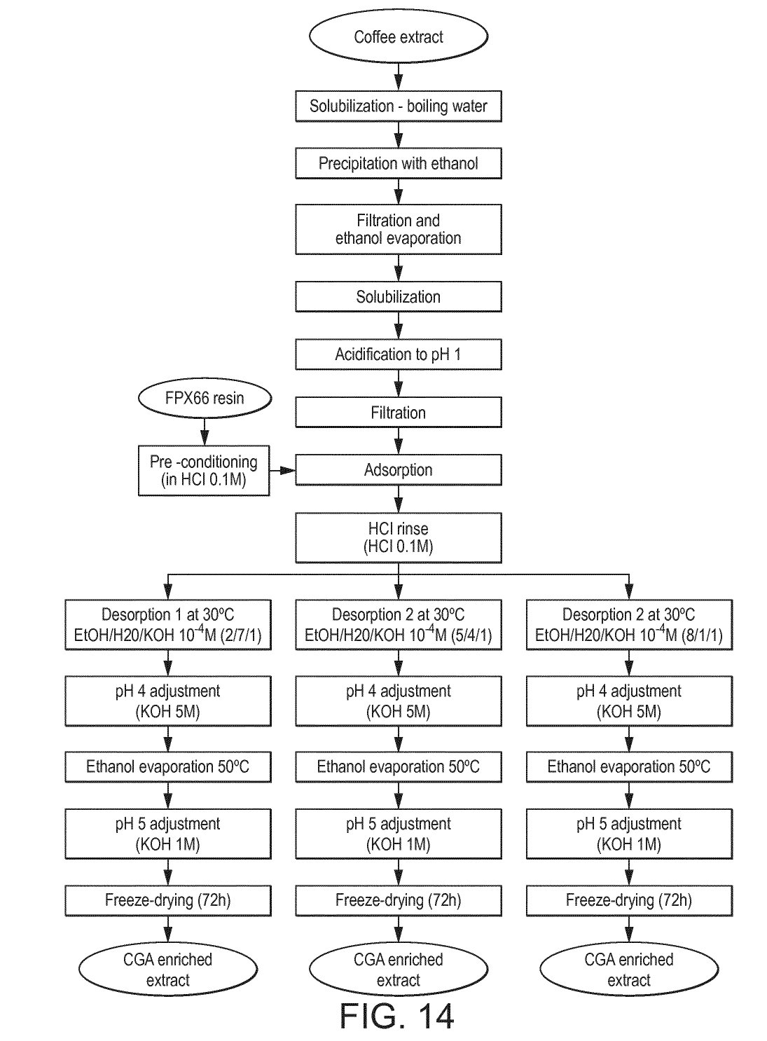

Desorption was performed in three successive steps with increasing ethanol ratio in the desorption mix. The protocol is displayed in FIG. 14. Three purified enriched fractions were obtained as follows:

Step 1—desorption with ethanol/water/KOH 20/70/10 (v/v/v);

Step 2—desorption with ethanol/water/KOH 50/40/10 (v/v/v);

Step 3—desorption with ethanol/water/KOH 80/10/10 (v/v/v).

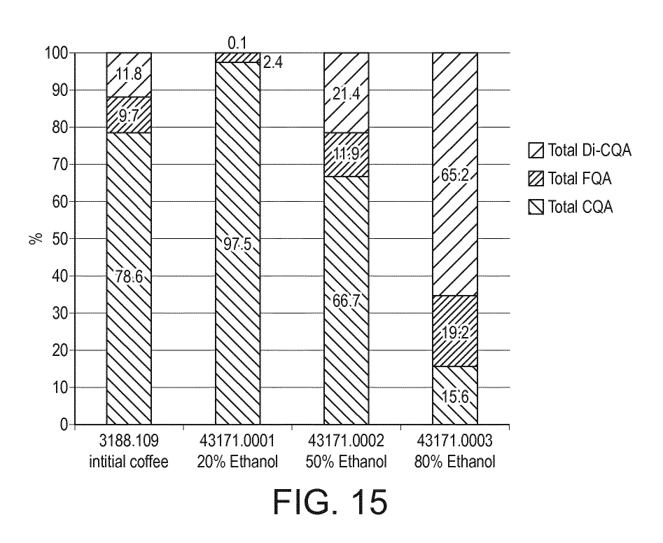

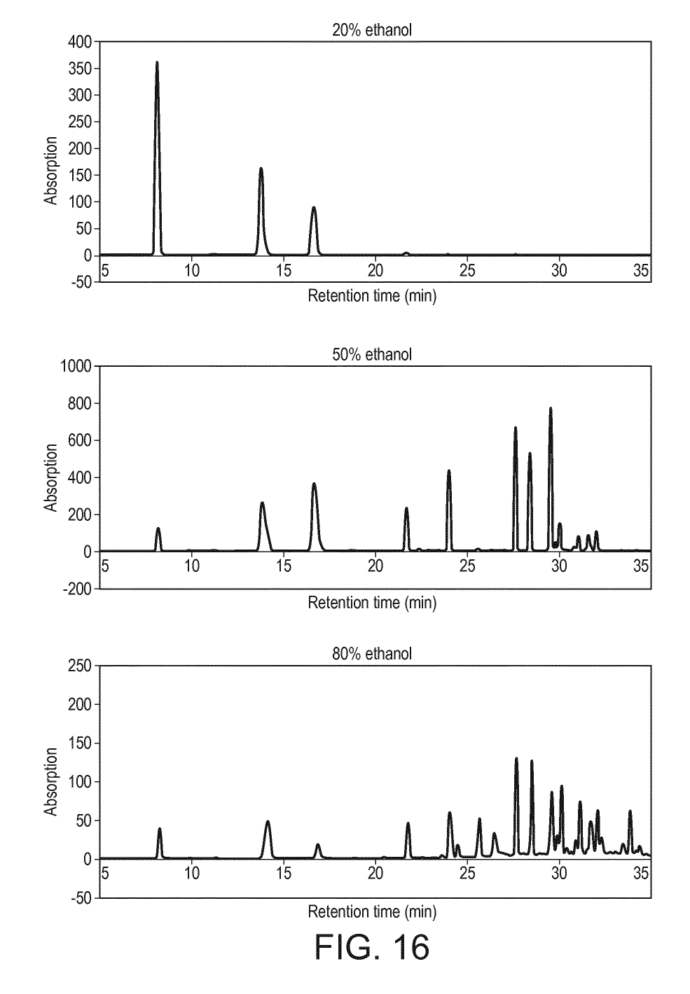

By applying a gradient for the desorption from the food grade resin three different enriched fractions were obtained. The chlorogenic acid composition of these enriched fractions was then assessed by HPLC-UV. FIG. 15 shows the relative contribution for the three major classes of CGA. FIG. 16 shows HPLC chromatograms of the three different enriched fractions.

The main findings are:
All enriched fractions contain about twice more polyphenols compared to the initial extract (i.e. 65-85% vs. 33%) as previously shown;
Desorption with 20% ethanol leads to an enriched fraction that contains 70.7% chlorogenic acids of which 97% are mono CQAs and 2.4% mono FQAs. Thus, an enriched fraction containing almost exclusively CQA is obtained at low ethanol ratio (i.e. 20/80);
Desorption with 50% ethanol leads to an enriched fraction that contains 85.7% chlorogenic acids with a profile close to that of the initial green coffee extract. Thus, an enriched fraction with a mixed composition of CQA, FQA and diCQA is obtained at intermediate ethanol ratio (i.e. 50/50);
Desorption with 80% ethanol leads to an enriched fraction that contains 65.1% chlorogenic acids of which 65% are the less polar di-CQA. Thus, a fraction enriched in apolar di-CGA is obtained at high ethanol ratio (i.e. (80/20).

This gradient application may be helpful in situations where it is desired to improve desorption of the various forms of chlorogenic acid compounds and enriched fractions may afterwards be combined to provide an isomeric balance of the CGA that resemples the isomeric balance of CGA originally in the coffee. The gradient desorption may also be useful in the case where a more specific composition is needed.

1. A process for providing a fraction enriched in polyphenols from a starting material, the process comprises the steps of:
(i) solubilizing the starting material in an aqueous solvent;
(ii) adjusting pH to below pH 3;
(iii) contacting the starting material with a chromatographic resin;
(iv) desorbing the polyphenols from the chromatographic resin to provide an eluate comprising the polyphenols;
(v) adjusting pH of the eluate to above pH 3.5; and
(vi) separating the eluate from the eluted polyphenols to obtain the fraction enriched in polyphenols.

2. The process according to claim 1, wherein the solubilized starting material obtained in step (i) is subjected to precipitation with an alcohol to provide a solid phase and a liquid phase, before the pH-adjustment in step (ii).

3. The process according to claim 2, wherein the liquid phase is subjected to an evaporation treatment to remove the alcohol from the liquid phase, before the pH-adjustment in step (ii).

4. The process according to claim 1, wherein the temperature during desorption is below 70° C.

5. The process according to claim 1, wherein the desorption eluent comprises a hydroxyl component combination with an aqueous base.

6. The process according to claim 1, wherein the starting material is a plant material.

7. A fraction enriched in polyphenols obtainable from the provided by a process comprising the steps of: (i) solubilizing the starting material in an aqueous solvent; (ii) adjusting pH to below pH 3; (iii) contacting the starting material with a chromatographic resin; (iv) desorbing the polyphenols from the chromatographic resin to provide an eluate comprising the polyphenols; (v) adjusting pH of the eluate to above pH 3.5; and (vi) separating the eluate from the eluted polyphenols to obtain the fraction enriched in polyphenols.

8. The fraction enriched in polyphenols according to claim 7, having a content of polyphenols increased by at least 70% compared to its initial level on a dry matter basis.

9. The fraction enriched in polyphenols according to claim 7, having a content of ethylesters of polyphenols below 20% (w/w) on a dry matter basis relative to the total content of polyphenols.

10. The fraction enriched in polyphenols according to claim 7, wherein the polyphenol is selected from the group consisting of a chlorogenic acid, caffeic acid and combinations thereof.

11. The fraction enriched in polyphenols according to claim 7, wherein the initial balance in chlorogenic acid of the plant extract is substantially maintained after purification.

12. A food comprising a fraction enriched in polyphenols obtained by a process for providing a fraction enriched in polyphenols from a starting material, the process comprises the steps of: (i) solubilizing the starting material in an aqueous solvent; (ii) adjusting pH to below pH 3; (iii) contacting the starting material with a chromatographic resin; (iv) desorbing the polyphenols from the chromatographic resin to provide an eluate comprising the polyphenols; (v) adjusting pH of the eluate to above pH 3.5; and (vi) separating the eluate from the eluted polyphenols to obtain the fraction enriched in polyphenols.

13. A food according to claim 12, wherein the food is a food ingredient.

14. A food according to claim 12 as a precursor for generating aroma, flavor and/or foaming.

15. (canceled)

16. The fraction according to claim 7, wherein the fraction is part of a pharmaceutical composition.
